(19) United States
(12) Patent Application Publication
(10) Pub. No.: US 2025/0195561 A1
(43) Pub. Date: Jun. 19, 2025

(54) A CATIONIC DENDRIMER FOR IMPROVING ANTIBIOTIC EFFICACY OF AN ANTIBIOTIC

(71) Applicant: GAMA HEALTHCARE LTD, Hertfordshire (GB)

(72) Inventor: Adrian FELLOWS, Hertfordshire (GB)

(21) Appl. No.: 18/840,845

(22) PCT Filed: Feb. 23, 2023
(86) PCT No.: PCT/EP2023/054587

§ 371 Date: Aug. 22, 2024

(30) Foreign Application Priority Data
Feb. 25, 2022 (GB) 2202666.0

(51) Int. Cl. A61K 31/785; A61K 31/407; A61K 31/7036; A61P 31/04

(52) U.S. Cl. CPC A61K 31/785; A61K 31/407; A61K 31/7036; A61P 31/04

(57) ABSTRACT

The present invention is directed to improving the antibiotic efficacy of antibiotics and to an antimicrobial composition with increased antibiotic efficacy.

The present invention is directed to improving the antibiotic efficacy of antibiotics and to an antimicrobial composition with increased antibiotic efficacy.

Antibiotics are the most important type of antibacterial agents for fighting infections by microorganisms and antibiotic medications are widely used in the treatment and prevention of such infections. Since their introduction, antibiotics have revolutionised the field of infectology and their availability and efficacy is vital for health care in humans and animals.

A wide range of antibiotics has been developed against infections by different microorganisms. However, the widespread use of antibiotics has led to the problem of antibiotic resistance, i.e. microorganisms becoming resistant to antibiotics. Infections due to antibiotic resistance are causing millions of deaths each year. Infections by antibiotic resistant microorganisms are more difficult to treat, require a higher dosage of antibiotics or alternative antibiotics.

In view of this, extensive research has been made in order to identify new antibiotics but the number of new antibiotic drugs developed in recent years is low.

An alternative approach to developing new antibiotic substances or increasing the dosage of known antibiotic substances could be developing means to increase the antibiotic efficacy of existing antibiotics. However, only few such approaches have been developed.

In light of the above, it was an object of the present invention to provide means for improving the antibiotic efficacy of antibiotics.

It was a further object of the present invention to provide antimicrobial compositions with increased antibiotic efficacy.

According to one aspect, said object is solved by a cationic dendrimer for improving the antibiotic efficacy of an antibiotic.

The present inventors have surprisingly found that cationic dendrimers can strongly increase the antibiotic efficacy of existing antibiotic substances.

Without wanting to be bound by any specific theory, the present inventors assume that this is at least partly due to the inhibition of the efflux pump mechanism in the microorganism.

The efflux pumps are proteins that are encoded by genes such as qacA and cepA and that are embedded in the bacterial cell plasma membrane. Their function is to recognise noxious, potentially damaging agents that have penetrated the cell wall and reached the periplasm or cytoplasm. The efflux pump then extrudes or expels the agent to the external environment before it reaches its target. Efflux pumps are therefore transporters of noxious compounds from within the bacterial cell to the external environment. This is achieved by using energy derived from adenosine triphosphate (ATP) or the proton motive force (pmf). So called ABC transporters directly use ATP whilst RND-type efflux pumps use a hydronium ion pH gradient. Efflux pump expression and enhancement can arise from chromosomal mutation or plasmid acquisition.

Study of the mechanisms of efflux pumps has given rise to an understanding of their function and subsequently, the means by which their function may be inhibited. Four main mechanisms of efflux pump inhibition have been identified. These are as follows.
1. Reduction of the bacterial cell access to ions such as Ca2+, which play cofactor roles in efflux pumps.
2. Inhibition of access to the energy provided by the proton-motive force (pmf).
3. Inhibition of enzymes that provide the hydronium ions required for maintenance of the pmf.
4. Competing with the invading noxious agent for access to the efflux pump, for example by non-specific blocking or coating of the bacterial envelope.

While the present invention is not generally limited to the choice the cationic dendrimers, in one embodiment the dendrimer is any of a generation 0 to generation 3 dendrimer or a combination of the same, such as a generation 0 dendrimer, a generation 1 dendrimer, a generation 2 dendrimer, and/or a generation 3 dendrimer.

In one embodiment, the dendrimer is of generation 1.

Dendrimers fall into many different types of chemical structures. While the present invention is not limited to any specific type of dendrimer, in one embodiment the dendrimer is any or a combination of polyamidoamine (PAMAM) dendrimers, quaternary ammonium functionalised poly(propylene imine), polylysine, dendrimers with surface groups based on a sugar.

In one embodiment, the dendrimer is a polyamidoamine (PAMAM) dendrimer.

In one embodiment, the dendrimer is a polyamidoamine (PAMAM) dendrimer of generation 1.

Particularly good results have been achieved, when the dendrimer is polyamidoamine (PAMAM) dendrimer, generation 1.0, primary amine surface.

Very good results have been achieved with the commercially available dendrimer.

Dendritech® polyamidoamine (PAMAM) dendrimer, generation 1.0, primary amine surface, water solution.

Family code: 121

Concentration: 27.41% w/w

Manufactured by: Dendritech®, Inc., 3110 Schuette Dr., Midland. MI 48642 USA

The present invention is also directed to an antimicrobial composition comprising a dendrimer as described above.

In one embodiment, the antimicrobial composition further comprises an antibiotic.

In one embodiment, the antimicrobial composition comprises at least one antibiotic selected from the group consisting of aminoglykosid antibiotics and β-lactam antibiotics.

While the antimicrobial composition according to the present invention is not generally limited to any specific antibiotic, particularly good results have been achieved when the antimicrobial composition comprises one or more of gentamicen and imipenem.

The present invention is also directed to the antimicrobial composition as described above for use in the treatment and/or prophylaxis of diseases.

More specifically, the present invention is directed to the antimicrobial composition as described above for use in the treatment and/or prophylaxis of infections.

The present invention is particularly useful in dealing with microorganisms, which have gained resistance to antibiotics. Such antibiotic resistant microorganisms are an enormous problem in the field of medicine and the present invention is particularly useful in fighting such antibiotic resistant microorganisms.

The present invention is therefore also directed to an antimicrobial composition as described above for use in the treatment and/or prophylaxis of infections by antibiotic resistant microorganisms.

While the present invention is not generally limited concerning the type of microorganism to be treated, particularly good results have been achieved for the antimicrobial composition described above for use in the treatment and/or prophylaxis of infections by E. coli, Staph aureus, Klebsiella pneumoniae, Streptococcus pneumoniae, Acinetobacter baumannii and Pseudomonas aeruginosa.

In particular, the antimicrobial composition described above is for use in the treatment and/or prophylaxis of infections by antibiotic resistant microorganism of the species E. coli, Staph aureus, Klebsiella pneumoniae, Streptococcus pneumoniae, Acinetobacter baumannii and Pseudomonas aeruginosa.

The present invention is also directed to a method for the treatment and/or prophylaxis of microbial infections using at least one dendrimer as described above or an antimicrobial composition as described above.

The present invention is further illustrated by the following non-limiting example.

### EXAMPLE

As set of experiments was conducted showing that the cationic dendrimers according to the present invention prove the antibiotic efficacy of antibiotics.

The following dendrimer was used in the experimental part:

Dendritech® polyamidoamine (PAMAM) dendrimer, generation 1.0, primary amine surface, water solution.

Family code: 121

Lot #: 1020-05-E1.0-PT-W

Concentration: 27.41% w/w

Manufactured by: Dendritech®, Inc., 3110 Schuette Dr., Midland. MI 48642 USA

### 1. Introduction

Antibiotic disc diffusion assays, 96-well broth dilution tests and confocal scanning laser microscopic (CLSM) techniques were employed to determine the ability of dendrimers to act as efflux pump inhibitors (EPI's) on the clinical Pseudomonas aeruginosa strain PS3 and the common research strain PA01.

Alanine β-naphthylamide (Ala-Nap) is a fluorescent compound that can aid in the search for new EPI's. Ala-Nap only fluoresces when inside the cell, therefore when efflux pumps are blocked an increase in fluorescence will be observed as more Ala-Nap will have accumulated inside the cell.

### 2. Test Procedures

### 2.1 Antibiotic Disc Diffusion Assay

The antibiotic disc diffusion assay was performed as described previously [1] to determine the antibiotic susceptibility of the two strains. Briefly, a standardised suspension of the two Pseudomonas strains was used to inoculate Iso-Sensitest agar (ISA) plates to yield semi-confluent growth of each organism. Antibiotic discs containing Gentamicin and Imipenem were applied to the inoculated plates, then incubated at 37° C. for 20 hours. Zones of inhibition were measured to the nearest millimetre.

### 2.2 96-Well Broth Dilution Testing

96-well broth dilution tests were carried out to determine the minimum inhibitory concentration (MIC) of antibiotics used in the disc diffusion assay, in the presence and absence of dendrimer. Briefly, a standard suspension of the two Pseudomonas strains was prepared in Mueller Hinton Broth (MHB), then added to varying concentrations of antibiotic in a 96-well plate. Tests were performed either in the presence or absence of dendrimer (final concentration 0.1%). 96-well plates were incubated at 37° C. for 24 hours, with optical density (620 nm) of cultures monitored over this period using a Multiskan EX plate reader (Thermo Labsystems). Final MIC values in the presence and absence of dendrimer were calculated using a modified Gompertz function as described previously [2].

### 2.3 Fluorescence Microscopy

Confocal laser scanning microscopy (CLSM) was employed in order to visualise the impact of the dendrimer on Pseudomonas efflux pumps. The assays were initiated by the addition of Ala-Nap (final concentration 64 mg/ml) to the cells present in the broth dilution testing (Section 2.2). Confocal laser scanning microscopy of the treated cells was performed at the Bio-imaging facility at the University of Huddersfield. A Zeiss LSM880 inverted confocal microscope was used for the imaging. Images were processed and the fluorescence intensity of cells was quantified using Zen 2.1 software (Zeiss Microscopy).

### 3 Results

### 3.1 Antibiotic Disk Diffusion Assay

Table 1 shows the zone of inhibition generated by each antibiotic during the disc diffusion assay. Strains PA01 and PS3 were more susceptible to Gentamicin than Imipenem.

### Zone of Inhibition (Mm±Stdev)

TABLE 1
Zone of inhibition assay results generated by various antibiotics
against Pseudomonas aeruginosa strains PA01 and PS3 (n = 2).

| Antibiotic | PA01 | PS3 |
|---|---|---|
| Gentamicin | 38 (±1.4) | 46 (±0.7) |
| Imipenem | 12 (±2.8) | 35 (±0.0) |

### 3.2 MIC Testing

In the absence of dendrimer the MIC for strain PAO1 and PS3 to Gentamicin was 4.64 and 1.45 mg/L respectively (Table 2). In the presence of dendrimer the MIC of Gentamicin to strains PA01 and PS3 decreased to 1.34 and 0.32 mg/L respectively (Table 2), indicating the dendrimers were reducing the MIC for this antibiotic by 71% and 78% respectively.

TABLE 2
Minimum inhibitory concentration of Gentamicin for strains
PA01 and PS3 in the presence and absence of dendrimer.

| | MIC (mg/L) | | | |
|---|---|---|---|---|
| | | Gentamycin + | Fold | Reduction |
| Organism | Gentamycin | Dendrimer | Change | (%) |
| PA01 | 4.64 | 1.34 | −3.46 | 71.12% |
| PS3 | 1.45 | 0.32 | −4.53 | 77.93% |

The MIC for strains PA01 and PS3 against Imipenem in the absence of dendrimer was 28.33 and 51.12 mg/L respectively (Table 3). In the presence of dendrimer both strains became highly susceptible to Imipenem with MIC values reducing to <0.011 mg/L, which signifies a >99.9% reduction in MIC for both strains (Table 3).

TABLE 3
Shows minimum inhibitory concentration of Imipenem for
strains PA01 and PS3 in the presence and absence of
dendrimer. A significant reduction in MIC for both strains
was observed in the presence of the dendrimer.

| | MIC (mg/L) | | | |
|---|---|---|---|---|
| | | Imipenem + | | Reduction |
| Organism | Imipenem | Dendrimer | Fold Change | (%) |
| PA01 | 28.33 | <0.011 | >−2575.45 | >99.96% |
| PS3 | 51.12 | <0.011 | >−4647.27 | >99.98% |

### 3.3 Confocal Microscopy

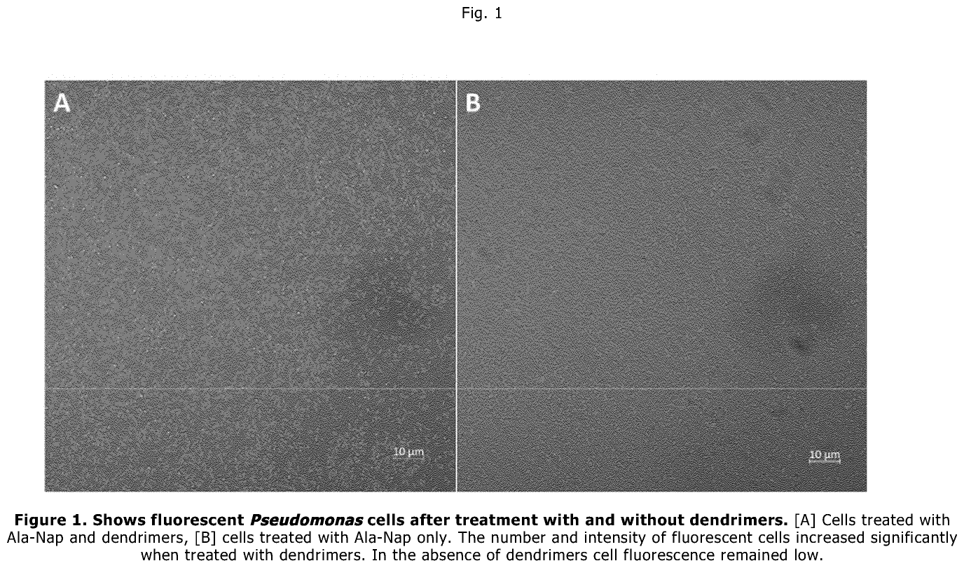

Following treatment with dendrimers the number and intensity of fluorescent cells were visualised using confocal laser scanning microscopy. FIG. 1 shows the fluorescent cells overlaid onto a bright-field image to differentiate between fluorescing and non-fluorescing cells. In the absence of dendrimer, very few cells were shown to be fluorescing (FIG. 1B). In contrast significantly more cells were fluorescing in the presence of dendrimer, suggesting efflux pumps were inhibited and demonstrating the mechanism by which MIC values were reduced during the broth dilution testing.

Fig. shows fluorescent Pseudomonas cells after treatment with and without dendrimers. [A] Cells treated with Ala-Nap and dendrimers, [B] cells treated with Ala-Nap only. The number and intensity of fluorescent cells increased significantly when treated with dendrimers. In the absence of dendrimers cell fluorescence remained low.

1. Cationic dendrimer for improving the antibiotic efficacy of an antibiotic.

2. Dendrimer according to claim 1, wherein the dendrimer is selected from the group consisting of a generation 0 dendrimer, a generation 1 dendrimer, a generation 2 dendrimer, a generation 3 dendrimer, and any combination thereof.

3. Dendrimer according to claim 1, wherein the dendrimer is of generation 1.

4. Dendrimer according to claim 1, wherein the dendrimer is selected from the group consisting of polyamidoamine (PAMAM) dendrimers, quaternary ammonium functionalised poly(propylene imine), polylysine, dendrimers with surface groups based on a sugar, and any combination thereof.

5. Dendrimer according to claim 1, wherein the dendrimer is a polyamidoamine (PAMAM) dendrimer.

6. Dendrimer according to claim 1, wherein the dendrimer is a polyamidoamine (PAMAM) dendrimer of generation 1.

7. Dendrimer according to claim 1, wherein the dendrimer is polyamidoamine (PAMAM) dendrimer, generation 1.0, primary amine surface.

8. Antimicrobial composition comprising a dendrimer according to claim 1.

9. Antimicrobial composition according to claim 9, further comprising an antibiotic.

10. Antimicrobial composition according to claim 9, wherein the antibiotic is selected from the group consisting of aminoglykosid antibiotics and β-lactam antibiotics.

11. Antimicrobial composition according to claim 8, further comprising one or more of gentamicen and imipenem.

12. (canceled)

13. (canceled)

14. (canceled)

15. (canceled)

16. A method for the treatment and/or prophylaxis of microbial infections using at least one dendrimer according to claim 1.

17. The method of claim 16, wherein the microbial infection is by an antibiotic resistant microorganism.

18. The method of claim 16, wherein the microbial infection is selected from the group consisting of E coli, Staph aureus, Klebsiella pneumoniae, Streptococcus pneumoniae, Acinetobacter baumannii, and Pseudomonas aeruginosa.

19. A method for treatment and/or prophylaxis of microbial infections using an antimicrobial composition comprising a dendrimer according to claim 1.

20. The method of claim 19, wherein the antimicrobial composition further comprises an antibiotic.

21. The method of claim 20, wherein the antibiotic is selected from the group consisting of aminoglykosid antibiotics and β-lactam antibiotics.

22. The method of claim 20, wherein the antibiotic is selected from the group consisting of gentamicen and imipenem.

23. The method of claim 19, wherein the infection is by antibiotic resistant microorganisms.

24. The method of claim 19, wherein the microbial infection is selected from the group consisting of E coli, Staph aureus, Klebsiella pneumoniae, Streptococcus pneumoniae, Acinetobacter baumannii and Pseudomonas aeruginosa.
